(19) United States
(12) Patent Application Publication
(10) Pub. No.: US 2016/0199145 A1
(43) Pub. Date: Jul. 14, 2016

(54) METHOD AND APPARATUS FOR MEASUREMENT OF INTRAMEDULLARY LENGTH WITH RADIOPAQUE MARKINGS

(71) Applicants: Biomet Manufacturing, LLC, Warsaw, IN (US); Biomet Sports Medicine, LLC, Warsaw, IN (US)

(72) Inventors: George J. Haidukewych, Orlando, FL (US); Cory Collinge, Nashville, TN (US); Frank A. Liporace, Englewood Cliffs, NJ (US); Daren Granger, Warsaw, IN (US); William Scott Van Dyke, Warsaw, IN (US); Anthony J. Metzinger, Winona Lake, IN (US); Joseph Angelo, Warsaw, IN (US)

(21) Appl. No.: 14/991,678

(22) Filed: Jan. 8, 2016

Related U.S. Application Data
(60) Provisional application No. 62/101,665, filed on Jan. 9, 2015.

(51) Int. Cl. A61B 90/00; A61B 17/16

(52) U.S. Cl. CPC A61B 90/06; A61B 17/164; A61B 17/1617; A61B 90/39; A61B 2090/3966; A61B 2090/062

(57) ABSTRACT

Systems and methods for measuring an intramedullary length for an orthopedic implant are described herein. A reamer system comprises a reamer shaft and a first reamer head. The reamer shaft comprises a proximal end having a drive coupler, a distal end having a reamer coupler, and markers located along the reamer shaft proximate the proximal end. The first reamer head comprises a head coupler configured to connect with the reamer coupler, and one or more cutting flutes located distal to the head coupler. The markers provide an indication of lengths of the system from a distal tip of the cutting flutes to each of the markers. The markers can be radiopaque. A method of employing the reamer system to measure the intramedullary length is described herein.

### CLAIM OF PRIORITY

This patent application claims the benefit of priority of U.S. Provisional Patent Application Ser. No. 62/101,665, entitled “Method and Feature for Identification and Measurement of Intramedullary Length with Radiopaque Markings,” filed on Jan. 9, 2015 (Attorney Docket No. 5394.B58PRV/BI01017PRV), which is hereby incorporated by reference herein in its entirety.

### BACKGROUND

Intramedullary nailing is an acceptable form of treating fractures. In order to place an appropriately sized implant into the bone, the canal must be reamed and measured for length. Currently, a guidewire is placed into the bone and a calibrated measuring device is placed in order to determine the length of the canal. This is followed by reaming the canal to the end of the wire. Fluoroscopy is used to ensure guidewire placement and correct placement of the measuring device in order to provide an accurate measurement.

U.S. Pat. No. 5,100,404 to Hayes describes the use of a radiopaque marker on a reamer guidewire to determine a length of a reamed hole. U.S. Pat. No. 5,514,137 to Coutts and U.S. Pat. No. 4,913,137 to Azer et al. also describe guidewire-assisted intramedullary nail implantation. U.S. Pat. No. 8,556,896 to Kitch et al. describes a ball tip guidewire having an enlarged opening that provides relief to facilitate removal of the guidewire. U.S. Pat. No. 5,989,260 to Yao describes a method for measuring an intramedullary depth using a graduated rod.

Modular reamer systems are routinely used to produce various sizes or diameters of reamed canals with fewer parts than would be possible with a non-modular system, e.g. a system where the reamer head is not removable from the reamer shaft. Particularly important in the orthopedic industry, modular reamer systems are useful for the speed and ease of use that is desirable in the operating room, as well as providing the operating surgeon with a wider range of tools while using fewer resources. However, a modular reamer system is dependent on being secure enough on the shaft to be safe to operate when rotated by a power tool, while simultaneously allowing for easy, quick, and safe removal of the reamer head. These goals can be at odds, as an overly secure reamer head will be difficult to remove, and one that is easy to remove may become uncoupled during use. In addition, any manual handling of a reamer head or other cutting tool presents the risk of damaging personal protective equipment and the exposure of the patient and surgeon to biohazards, as well as the risk of injury to the surgeon.

U.S. Pat. Nos. 6,783,533 and 6,332,886 to Green et al. describe attachable and detachable reaming heads having an aspiration tube. U.S. Pat. No. 5,163,790 to Vig describes a reamer with an interchangeable cutter head.

### OVERVIEW

The present inventors have recognized, among other things, that a problem to be solved can include the additional time and steps needed to measure an intramedullary nail length using a guidewire. Furthermore, the present inventors have recognized that a problem to be solved can include the potential inaccuracy of measuring an intramedullary nail length using a flexible guidewire. The present subject matter can help provide a solution to this problem, such as by eliminating the additional step of measuring an intramedullary nail length using a guidewire and, alternatively, measuring the intramedullary nail length using the rigid body of the reamer shaft in conjunction with interchangeable reamer heads of uniform length.

Systems and methods for measuring an intramedullary length for an orthopedic implant are described herein. A reamer system comprises a reamer shaft and a first reamer head. The reamer shaft comprises a proximal end having a drive coupler, a distal end having a reamer coupler, and markers located along the reamer shaft proximate the proximal end. The first reamer head comprises a head coupler configured to connect with the reamer coupler, and one or more cutting flutes located distal to the head coupler. The markers provide an indication of lengths of the system from a distal tip of the cutting flutes to each of the markers. The markers can be radiopaque. A method of employing the reamer system to measure the intramedullary length is described herein.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

FIG. 1 is a perspective view of a reamer system having a reamer shaft and a reamer head showing the proportional and overall lengths of the system, shaft and reamer head.

FIG. 2 is a close-up side view of a coupling mechanism for linking a slot of the reamer head to a projection of the reamer shaft.

FIG. 3 is a side view of the reamer head of FIG. 2 showing the slot for receiving the corresponding projection of the reamer shaft.

FIG. 4 is a perspective view of the reamer shaft of FIG. 1 without a reamer head attached to show the projection for connecting to a slot in a reamer head.

FIG. 5 is a close-up top view of the reamer shaft of FIG. 4 showing the projection extending from a reamer coupler.

FIG. 6 is a cross-sectional side view of the coupling mechanism of FIG. 2 showing the projection of the reamer shaft engaged with the slot of the reamer head and a guidewire.

FIG. 7 is a side view of another embodiment of a coupling mechanism for a reamer shaft and a reamer head.

FIG. 8 is a cross-sectional view of the coupling mechanism of FIG. 7 showing a finger body and socket interface.

FIG. 9 is a perspective view of a female coupler connected to a distal end of a reamer shaft as used in the coupling mechanism of FIG. 7.

FIG. 10 is a cross-sectional view of the female coupler and the distal end of the reamer shaft of FIG. 9 showing a distal anti-rotation feature and an axial locking feature.

FIG. 11 is a perspective view of the reamer head connected to the finger body as used in the coupling mechanism of FIG. 8.

FIG. 12 is a cross-sectional view of the reamer head and the finger body of FIG. 11 showing flexible fingers and a hex feature.

FIG. 13 is a perspective view of a removal tool about to be inserted in between a reamer head and a reamer shaft.

FIG. 14 is a perspective view of the removal tool advanced into a slot between the reamer head and the reamer shaft of FIG. 13.

FIG. 15 is a perspective view of the removal tool of FIG. 14 fully advanced into the slot to pull the reamer head away from the reamer shaft.

FIG. 16 is a perspective view of the removal tool for removing a reamer head from a reamer shaft as incorporated into a receiver box.

FIG. 17 is a perspective view of another example of a reamer system in which a reamer coupler of a reamer shaft includes a slot for receiving the removal tool.

FIG. 18 is a perspective view of the reamer shaft of FIG. 17 with the reamer head fully seated.

FIG. 19 is a cross-sectional view of fingers of the reamer head fully seated within the slot of the reamer coupler.

FIG. 20 is a perspective view of the reamer shaft and the reamer head of FIG. 18 in which the removal tool is partially advanced to partially separate the reamer head.

FIG. 21 is a perspective view of the reamer shaft and the reamer head of FIG. 20 in which the removal tool is fully advanced to fully separate the reamer head.

FIG. 22 is a cross-sectional view of the fingers of the reamer head being displaced from the slot of the reamer coupler via arms of the removal tool.

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

### DETAILED DESCRIPTION

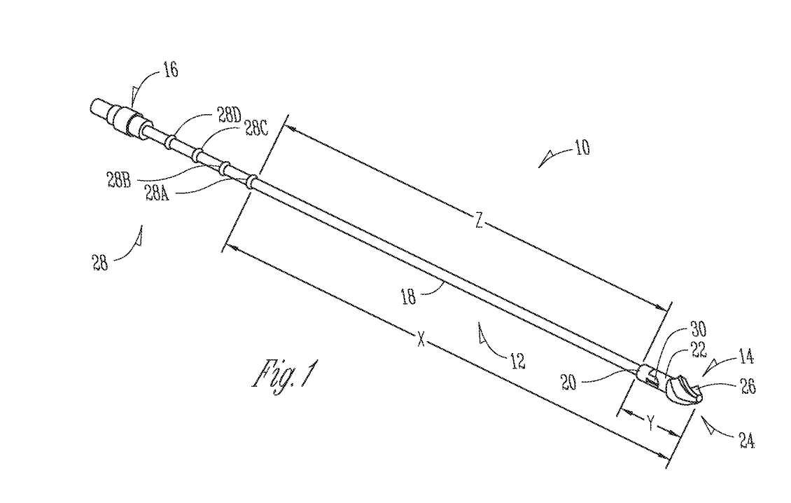

FIG. 1 is a perspective view of a reamer system 10 having a reamer shaft 12 and a reamer head 14 showing the proportional and overall lengths of the system 10, shaft 12 and reamer head 14. The reamer shaft 12 can include a proximal drive coupler 16, an elongate shaft body 18 and a reamer coupler 20. The reamer head 14 can include a head coupler 22 and a reamer body 24, which can include cutting lobes 26. Reamer shaft 12 can also include markers 28, which can be used to reference the distance to the distal tip of the reamer head 14.

The proximal drive coupler 16 can be used to connect the reamer system 10 to a drive mechanism that can provide rotational input to the reamer system 10. For example, a typical drive mechanism may comprise a rotary input device such as a hand-held power tool or a drill. The drive coupler 16 may comprise any suitable device as is known in the art. In an example, the drive coupler may comprise a hex head that can be inserted into a socket of a rotary input device.

The elongate shaft body 18 can be a tube or any other elongate body that can allow the reamer head 14 to be extended into a bone. The elongate shaft body 18 can be hollow to allow for the passage of a guidewire therethrough. The elongate shaft body 18 can be one single piece or a plurality of interconnected components. In an example, shaft body 18 is comprised of nitinol (nickel titanium) metal alloy. In other examples, shaft body 18 can be fabricated from other biocompatible materials, such as metals, alloys or polymers.

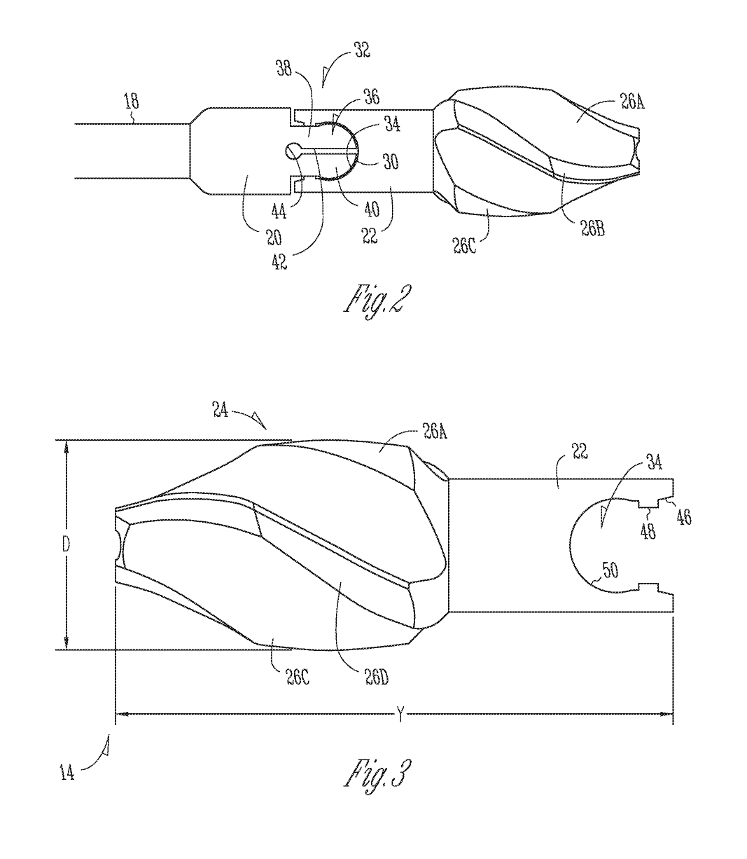

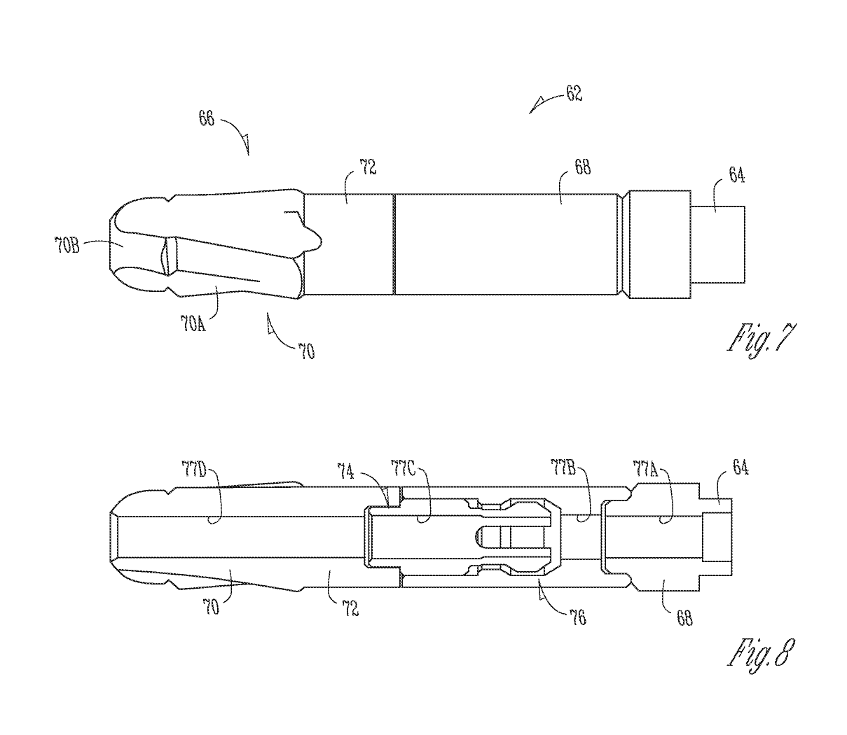

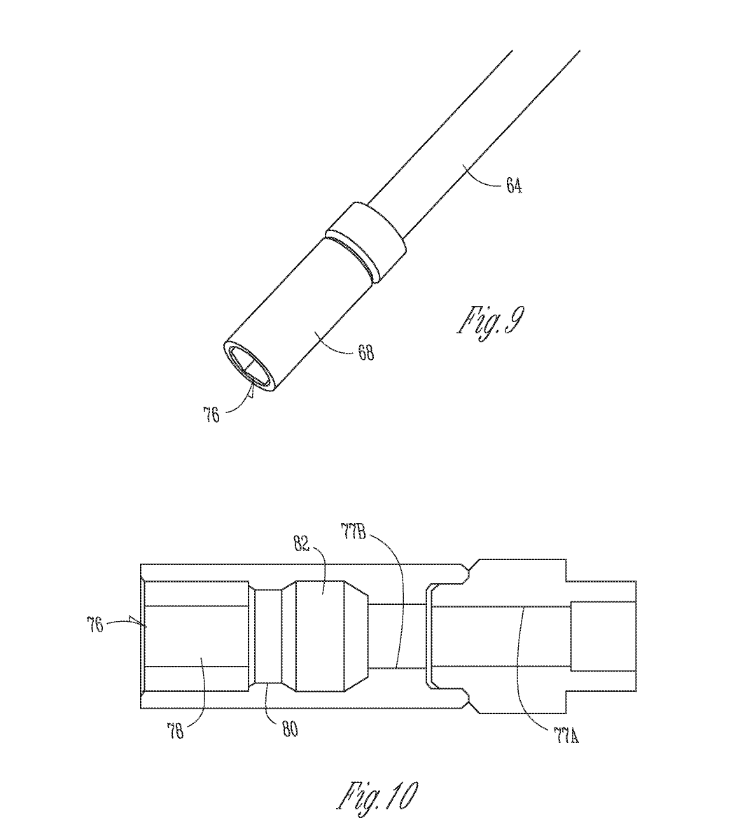

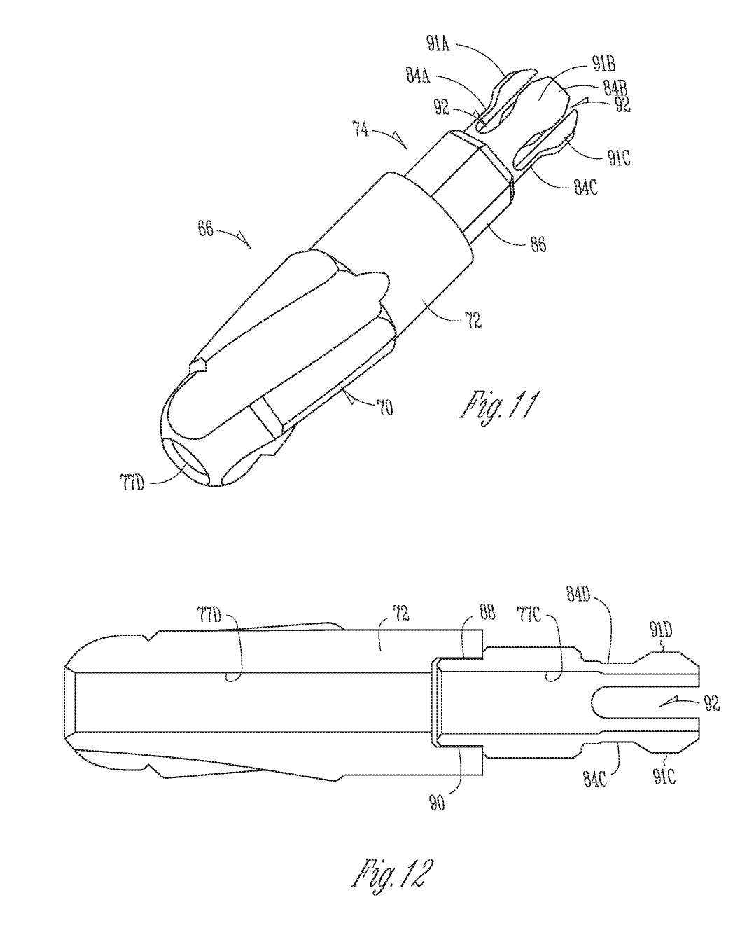

The reamer coupler 20 can provide an interface for connecting to the reamer head 14. The reamer head 14 can be configured to be repeatably coupled to the reamer coupler 20. For example, the head coupler 22 can be configured to releasably engage with the reamer coupler 20. As is discussed in greater detail below with reference to FIGS. 2-6, the head coupler 22 and the reamer coupler 20 can comprise a projection and slot interface. In another example, as discussed below with reference to FIGS. 7-12, the head coupler 22 and the reamer coupler 20 can comprise a finger and socket interface. However, the reamer head 14 and the reamer shaft 12 can be coupled using other suitable couplers.

The cutting lobes 26 of the reamer body 24 can be configured to provide side cutting and end cutting to remove bone material, e.g. cortical and cancellous bone, from a bone to provide a passage for an intramedullary nail. The markers 28 can be used to determine how far the reamer head 14 has been or is advanced into the reamed bone passage. In the example shown, four markers 28A, 28B, 28C and 28D are included on the reamer shaft 12. In other examples, fewer or more markers can be used.

The length X represents the combined length of the reamer system 10 from the marker 28A to the distal end of cutting lobes 26. Length Y represents the combined length of the head coupler 22 and the reamer body 24, including the cutting lobes 26. Length Z represents the length of elongate shaft body 18 from marker 28A to the distal end 30 of the reamer coupler 20. Length X can be the equivalent of length Y plus length Z, minus any overlap produced by engagement of reamer coupler 20 and head coupler 22. Length X increases by an amount corresponding to the distance between adjacent markers moving from marker 28A to marker 28B, etc.

The reamer head 14 may comprise any one of a plurality of reamer heads 14 having a variety of different reaming diameters. However, each reamer head 14 has the same length Y. As such, the distance from the distal end of cutting lobes 26 to the markers 28 is the same for each reamer head 14, regardless of the cutting diameter.

The markers 28 can include a plurality of markers 28A, 28B, 28C and 28D. Each marker 28A-28D can be spaced a predetermined distance from an adjacent marker with the distance between each marker corresponding to an increase in size of intramedullary nails. Thus, as each subsequent marker 28A-28D is advanced into a reamed intramedullary canal, it is understood that the required or desired length of an intramedullary nail increases by an incremental length corresponding to a predetermined or commercially available intramedullary nail size.

The reamer shaft 12 can be provided with indicia for each marker 28 that provides a written indication or description of the length X. For example, at each marker 28, the reamer shaft 12 can be etched with numbers representing the magnitude of X at a specific one of the markers 28. Markers 28 themselves can comprise radiopaque markers that are visible using fluoroscopy or X-ray systems and methods. Markers 28 can comprise suitably large projections that can be visible when reamer shaft 12 is viewed through tissue using a fluoroscopy or X-ray system. For example, markers 28 should be large enough to be viewed given the generally low resolution of fluoroscopy and X-ray systems and the obfuscation that tissue produces in fluoroscopic and X-ray images. In one example, markers 28 comprise rings circumscribing reamer shaft 12 and having a height of approximately 5 mm and a width along the length of the reamer shaft 12 of approximately 100 mm. In other examples, partial rings or other shapes can be used. However, markers may have a height in the range of approximately 2 mm to approximately 10 mm and can extend over a distance less than or greater than 100 mm.

FIG. 2 is a close-up side view of a coupling mechanism 32 for linking a slot 34 of the reamer head 14 to a projection 36 of the reamer coupler 20. The projection 36 can extend axially from neck 38 to form the distal end 30 of the reamer shaft 12 used to determine length X. However, distal end 30 for the purposes of determining length X can be located at the distal end of the portion of reamer coupler 20 connected to shaft body 18, rather than the distal end of flange 36 extending therefrom. The reamer coupler 20 can include a semi-circular or bulbous portion 40 extending from the projection 36. In a first radial dimension (up and down with respect to the orientation of FIG. 2), projection 36 can have a width that is larger closer to the distal end 30 of the reamer shaft 12 than at a location more proximal. For example, the thickness of the projection 36 at bulbous portion 40 can be greater than the thickness of the neck 38. As such, the projection 36 provides an axial ledge for engaging the slot 34 of the reamer head 14. In a second radial dimension (in and out of the plane of FIG. 2), the projection 36 can also extend radially across reamer coupler 20 (as shown in FIG. 5) so as to allow the projection 36 to provide rotational input, or torque, to the reamer head 14.

In an example, the projection 36 includes a first slit 42 that extends into the distal tip 30 of reamer coupler 20. The first slit 42 can allow projection 36 to flex in the first radial dimension. The first slit 42 can be used to impart tension to the head coupler 22. The first slit 42 can include a bore 44 to provide stress relief.

The reamer head 14 can include the slot 34 for engaging the projection 36. The slot 34 can have an opposite or corresponding shape to the projection 36 in the first and second radial dimensions to provide the desired axial and circumferential engagement and provide the corresponding tensile/compressive and rotational/torque force transmission capabilities. The cutting lobes 26 can be shaped to provide side and end cutting.

FIG. 3 is a side view of the reamer head 14 of FIG. 2 showing the slot 34 for receiving the corresponding projection 36 of the reamer shaft 12. The slot 34 can include a proximal entry 46, a neck 48 and a distal cavity 50. The proximal entry 46 can provide an opening for receiving the projection 36 of reamer coupler 20. The neck 48 can provide a minimum diameter portion of the slot 34 that engages the projection 36 to prevent axial displacement therebetween. The distal cavity 50 can provide a space for receiving the projection 36. The distal cavity 50 can have a larger radial dimension than the neck 48 in order to permit the axial engagement. The proximal entry 46, the neck 48 and the distal cavity 50 can have a uniform cross-section in the second radial dimension through the head coupler 22 in order to provide the corresponding axial and circumferential force transfer.

Cutting lobes 26 can comprise a plurality of lobes 26A, 26B, 26C and 26D that have generally axially extending edges for providing side cutting and generally radially extending edges at the distal tip for providing leading end cutting.

As mentioned the distance Y from the distal most end of reamer head 14 at the cutting lobes 26 to the proximal most end of head coupler 22 is the same for a plurality of different reamer heads having a plurality of different cutting lobes. For example, all of the different reamer heads 14 that can be used with reamer system 10 can have the same head coupler 22, including the same proximal entry 46, neck 48 and distal cavity 50. However, each reamer body 24 can be different in cutting diameter D. Thus, regardless of the specific reamer head 14 connected to reamer shaft 12, the distance X (FIG. 1) will be the same.

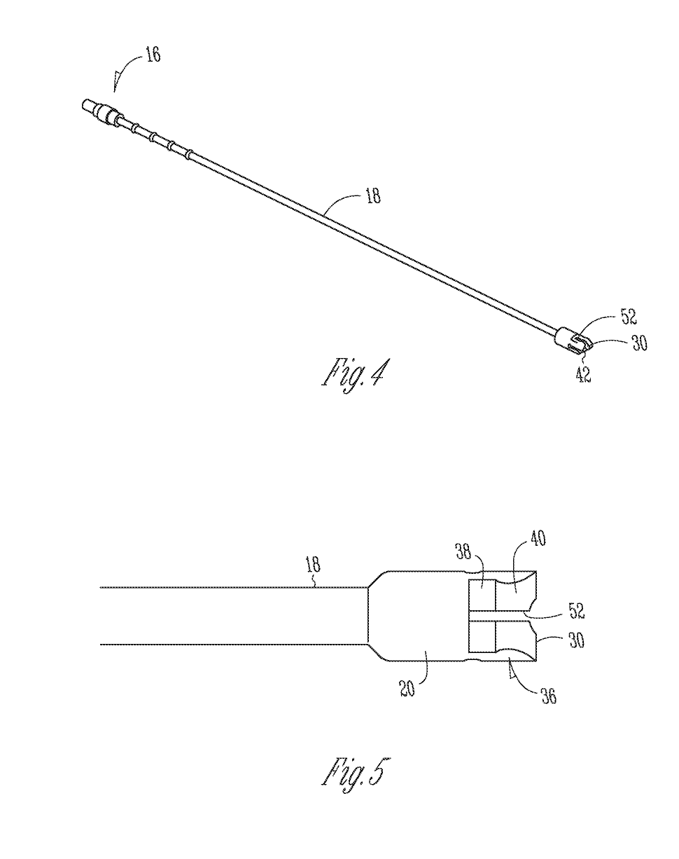

FIG. 4 is a perspective view of the reamer shaft 12 of FIG. 1 without the reamer head 14 attached to show the projection 36 for connecting to the slot 34 in the reamer head 14. As can be seen in FIG. 4, the projection 36 includes the distal end 30 into which the first slit 42 extends. The projection 36 also includes a second slit 52, which extends perpendicular to the first slit 42.

FIG. 5 is a close-up top view of the reamer shaft 12 of FIG. 4 showing the projection 36 extending from the reamer coupler 20. The reamer coupler 20 comprises a body that can be attached to the shaft body 18 such as by press fit or interference fit. The reamer coupler 20 can also be connected to the shaft body 18 by other means such as threaded engagement, set screws or spring pins. For example, in various examples, the shaft body 18 can be fabricated from nitinol, which can be difficult to join by metallurgical means such as welding. As such, mechanical fastening means can be desirable.

The neck 38 extends from a portion of the reamer coupler 20 connected to shaft body 18. As shown, neck 38 and bulbous portion 40 can extend all the way across reamer coupler 20 but for the presence of second slit 52. Bulbous portion 40 is configured to mate with the distal cavity 50 of the slot 34. The neck 38 can be configured to mate with the neck 48 of the slot 34. The first slit 42 and the second slit 52 allow the projection 36 to flex to facilitate coupling and removal of the projection 36 with the slot 34.

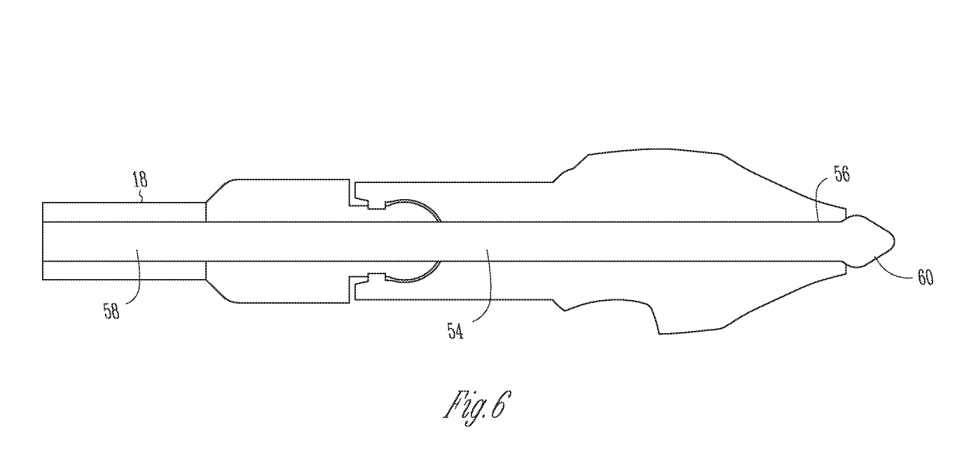

FIG. 6 is a cross-sectional side view of the coupling mechanism 32 of FIG. 2 showing the projection 36 of the reamer shaft 12 engaged with the slot 34 of the reamer head 14. FIG. 6 also shows a guidewire 54 extending through internal lumen 56 in the reamer head 14 and internal lumen 58 in shaft body 18. Guidewire 54 includes distal button 60. The guidewire 54 can be inserted into a bone prior to reamer system 100 is used to ream a passage for an implant, such as an intramedullary nail. A pilot hole for guidewire 54 can be produced using a small diameter drill bit or the like. After the guidewire 54 is positioned, reamer system 100 can be used to produce the passage, such as by using reamer heads 14 having progressively larger diameters D until the desired passage diameter for the desired implant is achieved. Each reamer head 14 is advanced into the bone along the guidewire 54 until a desired depth is achieved, which can be determined using markers 28 (FIG. 1). Distal button 60 can be used as a stop to limit advancement of each reamer head 14. Distal button 60 can also be used to withdraw each reamer head 14 from the bone such as by pulling on guidewire 54 in the proximal direction.

FIG. 7 is a side view of another embodiment of a coupling mechanism 62 for a reamer shaft 64 and a reamer head 66. The female coupler 68 can be joined to the distal end of the reamer shaft 64 via any suitable means. For example, in some embodiments where the reamer shaft 64 is comprised of a material that is difficult to weld, such as nitinol, the female coupler 68 can be press fit of force fit onto the distal end of the reamer shaft 64. In other examples, female coupler 68 and reamer shaft 64 can be monolithically formed from a single continuous body of material.

The reamer head 66 can include a lobe section 70 and a head coupler 72. Lobe section 70 can include side cutting lobes 70A and end cutting lobes 70B. The reamer head 66 can be connected to the female coupler 68 via an internal finger body 74, shown in FIG. 8, extending from the head coupler 72.

FIG. 8 is a cross-sectional view of the coupling mechanism 62 of FIG. 7 showing finger body 72 interfacing with a socket 76 of the female coupler 68. The reamer shaft 64, the female coupler 68, the finger body 72 and the reamer head 66 can include internal lumens 77A, 77B, 77C and 77D respectively, for receiving a guidewire.

FIG. 9 is a perspective view of the female coupler 68 connected to a distal end of the reamer shaft 64 as used in the coupling mechanism of FIG. 7. Female coupler 68 can include socket 76 for receiving finger body 74.

FIG. 10 is a cross-sectional view of the female coupler 68 and the distal end of the reamer shaft 64 of FIG. 9 showing a distal anti-rotation feature 78 and an axial locking feature 80 of socket 76. Socket 76 can also include proximal cavity 82.

FIG. 11 is a perspective view of the reamer head 66 connected to the finger body 74 as used in the coupling mechanism 62 of FIG. 7. Finger body 74 includes flexible fingers 84A, 84B, 84C and 84D and a hex feature 86. Reamer head 66 includes lobe section 70 extending from the head coupler 72 and internal lumen 77D.

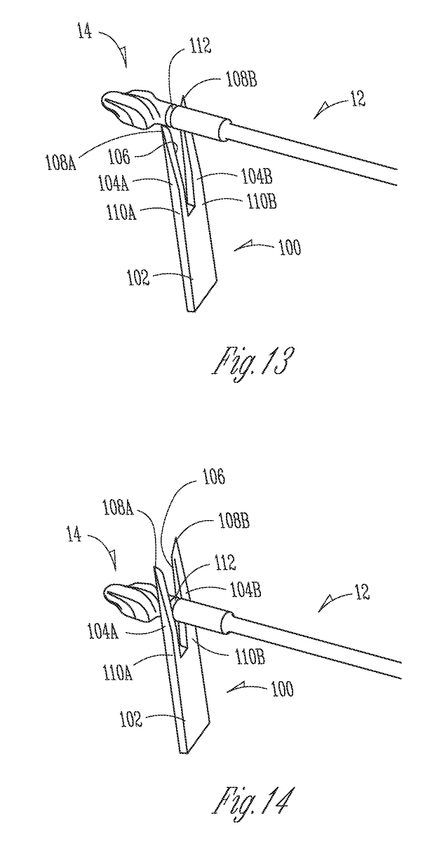

FIG. 12 is a cross-sectional view of the reamer head 66 and finger body 74 of FIG. 11 showing flexible fingers 84A, 84B, 84C and 84D and a hex feature 86. The head coupler 72 of the reamer head 66 can include a bore 88 for receiving shaft 90 of finger body 74. The bore 88 and the shaft 90 can form a press fit or interference fit to retain the finger body 74 within the reamer head 66. In other examples, reamer head 66 and finger body 74 can be monolithically formed from a single continuous body of material.

With continuing reference to FIGS. 8, 10 and 12, the hex feature 86 can comprise a circumferentially lobed cross-section that prevents rotation when seated within the anti-rotation feature 78 of the socket 76. In the depicted example, hex feature comprises a hexagonal feature having six planar facets that mate with six corresponding planar surfaces in the anti-rotation feature 78. However, other key shapes having different circumferentially lobed cross-sections can be used, such as star-shaped or octagonal.

The fingers 84A-84D can extend proximally from the hex feature 86. The fingers 86A-86D can be cantilevered from hex feature 86 in order to facilitate flexibility of each finger. The hex feature 86 is positioned distally from fingers 84A-84D in order to allow the fingers to be advanced into the socket 76 first and flex as they are extended past axial locking feature 80. The locking feature 80 can comprise a ridge circumscribing the socket 76.

Each finger 84A-84D includes a flange 91A-91D, respectively, that can be seated in the proximal cavity 82. The fingers 84A-84D are separated by cutouts 92 that can provide space to allow each finger to flex. Thus, as fingers 84A-84D are advanced into the socket 76, the fingers flex to allow the flanges 91A-91D to pass over the axial locking feature 80. Thereafter, during use, the presence of a guidewire within the lumen 77C will prevent the fingers 84A-84D from flexing and to undesirably allow the reamer head 66 to separate from the reamer shaft 64.

The fingers 84A-84D can only be fully inserted into the socket 76 when the hex feature 86 is circumferentially aligned with the anti-rotation feature 78. When the hex feature 86 and the anti-rotation feature 78 are circumferentially aligned and axially engaged, relative rotation between the reamer head 66 and the reamer shaft 64 is prevented.

FIG. 13 is a perspective view of a removal tool 100 being inserted in between a reamer head 14 and a reamer shaft 12. Removal tool 100 comprises a wedge shaped fork that can be driven between reamer head 14 and reamer shaft 12 in order to facilitate removal of the reamer head 14 from the reamer shaft 12.

Removal tool 100 can include base 102, first prong 104A and second prong 104B. First prong 104A and second prong 104B together can form removal slot 106. Distal ends 108A and 108A of first and second prongs 104A and 104B, respectively, can be narrower, or have a smaller thickness in the axial direction of reamer shaft 12, than proximal ends 110A and 110B of first and second prongs 104A and 104B, respectively. Distal ends 108A and 108BA are thin in order to facilitate insertion into a slot 112 that is formed in the break line between reamer head 14 and reamer shaft 12. Removal slot 106 can also become narrower as it extends from distal ends 108A and 108B to proximal ends 110A and 110B.

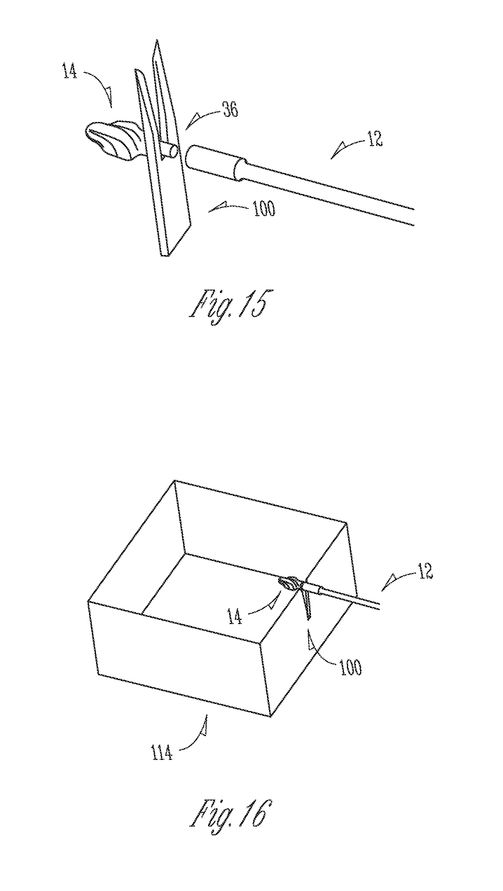

FIG. 14 is a perspective view of the removal tool 100 advanced into the slot 112 between the reamer head 14 and the reamer shaft 12 of FIG. 13. The thickness of prongs 104A and 104B grows as they advance from distal ends 108A and 108B to proximal ends 110A and 110B. As such, as removal tool 100 is advanced into slot 112, reamer head 14 is forced further away from reamer shaft 12.

FIG. 15 is a perspective view of the removal tool 100 of FIG. 14 fully advanced into the slot 112 to pull the reamer head 14 away from the reamer shaft 12. With reference to FIG. 3, as removal tool 100 is advanced into slot 112, upper and lower portions of projection 36 cane squeezed together via flexing of neck 38 into itself at slits 42 and/or 52, which allows bulbous portion 40 to squeeze down into neck 48 of head coupler 22. As removal tool 100 is further advanced into slot 112, bulbous portion 40 is advanced past neck 48 as slit 42 is squeezed together. Thus, reamer head 14 is freed from reamer shaft 12. The removal tool 100 can be used to provide a mechanical advantage in pulling reamer head 14 from reamer shaft 12 via wedge action. Separately or in combination with the wedge action, reamer tool 100 can be used to mechanically actuate a coupling mechanism, e.g. coupling mechanism 32, to allow for reamer head 14 to be removed from reamer shaft 12 with little or no resistance.

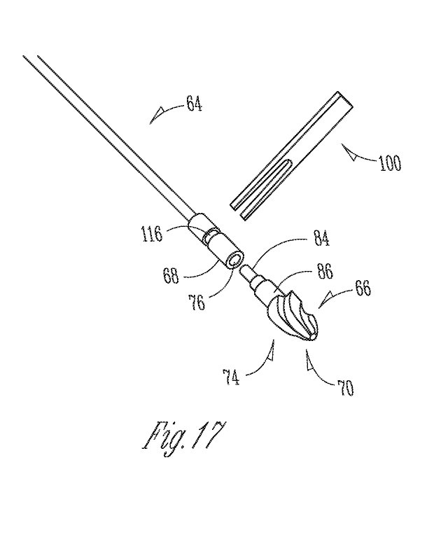

FIG. 16 is a perspective view of removal tool 100 for removing the reamer head 14 from the reamer shaft 12 as incorporated into a receiver box 114. In the example shown, removal tool 100 can be incorporated into, or otherwise attached to, receiver box 114. Receiver box 114 captures the reamer head 14 as it is withdrawn and separated from reamer shaft 12. In the example shown, receiver box 114 comprises a five-sided rectilinear compartment that allows the removed reamer head 14 to drop into the enclosure once freed from reamer shaft 12. Reamer head 14 is thus freed to fall into receiver box 82 whereby it can be retrieved and cleaned for future use. Other shaped retainers, such as cylindrical buckets, can also be used.

FIG. 17 is a perspective view of another example of a coupling device 62 in which the reamer coupler 68 includes a slot 116 for receiving the removal tool 100. The reamer head 66 includes fingers 84 extending from hex feature 86 as discussed above. Fingers 84 are configured to extend into socket 76 by flexing around the axial locking feature 80. Removal tool 100 can be inserted into slot 116 to squeeze the fingers 84 together to allow the reamer head 66 to be removed from the socket 76.

FIG. 18 is a perspective view of the reamer shaft 64 of FIG. 17 with the reamer head 66 fully seated. As discussed, the fingers 84 prevent reamer head 66 from being axially displaced from the reamer coupler 68.

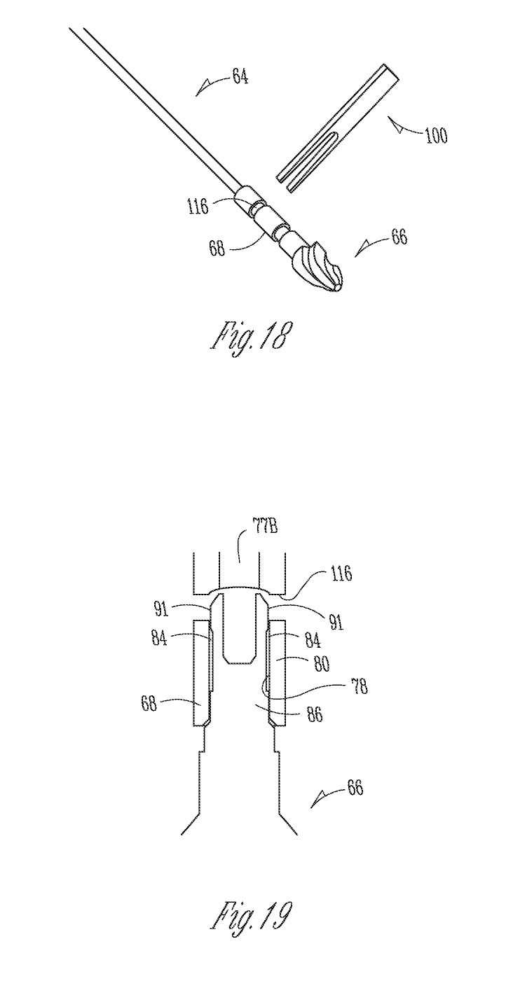

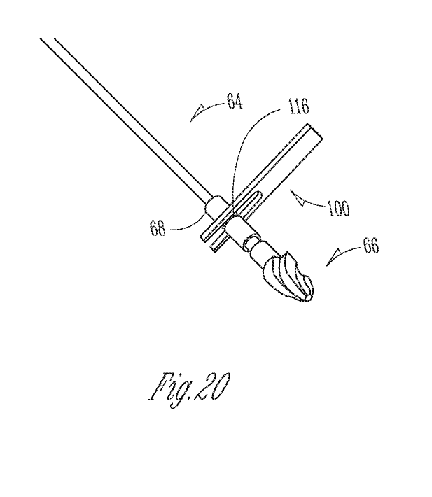

FIG. 19 is a cross-sectional view of the fingers 84 of the reamer head 66 fully seated within the slot 116 of the reamer coupler 68. Flanges 91 of fingers 84 are disposed proximal to the axial locking feature 80 such that reamer head 66 cannot be distally displaced. Flanges 91 are aligned with slot 116 to allow removal tool 100 to be able to interface with fingers 84.

FIG. 20 is a perspective view of the reamer shaft 64 and the reamer head 66 of FIG. 18 in which the removal tool 100 is partially advanced into slot 116 to partially separate the reamer head 66.

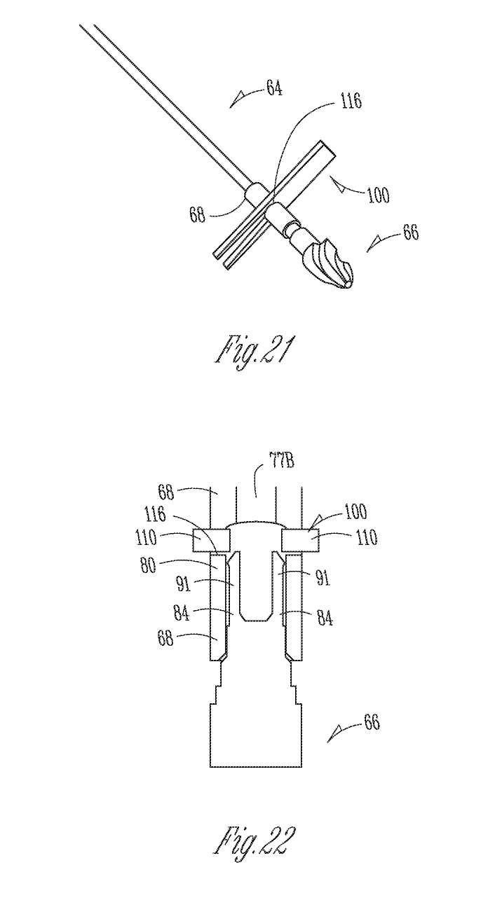

FIG. 21 is a perspective view of the reamer shaft 64 and the reamer head 66 of FIG. 20 in which the removal tool 100 is fully advanced to allow for full separation of the reamer head 66.

FIG. 22 is a cross-sectional view of the fingers 84 of the reamer head 66 being displaced from the slot 116 of the reamer coupler 68 via proximal ends 110 of the prongs 104 of the removal tool 100. Proximal ends 110 squeeze flanges 91 radially inward so that fingers 84 can be drawn into axial locking feature 80, thereby allowing reamer head 66 to be pulled out of socket 76. Thus, removal tool 100 allows reamer head 66 to be removed without a surgeon or other technician having to apply a substantial amount of human-generated force to the reamer head, which can potentially give rise to injury such as due to the cutting lobes.

### Various Notes & Examples

Example 1 can include or use subject matter, such as an apparatus for measuring an intramedullary length for an orthopedic implant, the system comprising: a reamer shaft comprising: a proximal end having a drive coupler a distal end having a reamer coupler; and markers located along the reamer shaft proximate the proximal end; and a first reamer head comprising: a head coupler configured to connect with the reamer coupler; and one or more cutting flutes located distal to the head coupler; wherein the markers provide an indication of lengths Of the system from a distal tip of the cutting flutes to each of the markers.

Example 2 can include, or can optionally be combined with the subject matter of Example 1, to optionally include a first reamer head comprising: a first length corresponding to the length of the system minus a length of the reamer shaft; and a first cutting diameter.

Example 3 can include, or can optionally be combined with the subject matter of Example 2 to optionally include a second reamer head comprising: a second length approximately equal to the first length; and a second cutting diameter different from the first cutting diameter.

Example 4 can include, or can optionally be combined with the subject matter of one or any combination of Examples 1-3 to optionally include a reamer coupler comprising a bulbous projection; and the head coupler comprises a slot shaped to inversely match the bulbous projection.

Example 5 can include, or can optionally be combined with the subject matter of Example 4 to optionally include a bulbous projection includes a split.

Example 6 can include, or can optionally be combined with the subject matter of one or any combination of Examples 1-5 to optionally include a reamer coupler comprising a socket having a ridge; and a head coupler comprising a plurality of flexible fingers configured to axially engage the ridge of the socket.

Example 7 can include, or can optionally be combined with the subject matter of Example 6 to optionally include a reamer coupler further comprising a circumferentially lobed extension of the socket; and a head coupler further comprising a key from which the plurality of flexible fingers extend, the key shaped to mate with the lobed extension and prevent relative rotation therebetween.

Example 8 can include, or can optionally be combined with the subject matter of one or any combination of Examples 1-7 to optionally include a reamer shaft and a first reamer head including axially aligned lumens.

Example 9 can include, or can optionally be combined with the subject matter of one or any combination of Examples 1-8 to optionally include a reamer coupler comprising a separate piece from the reamer shaft mechanically joined to the reamer shaft.

Example 10 can include, or can optionally be combined with the subject matter of one or any combination of Examples 1-9 to optionally include markers that are radiopaque.

Example 11 can include, or can optionally be combined with the subject matter of Example 10 to optionally include markers comprising a plurality of circumferential ribs extending from the reamer shaft.

Example 12 can include, or can optionally be combined with the subject matter of one or any combination of Examples 1-11 to optionally include a removal tool comprising: a forked wedge that is shaped to be inserted into a gap between the reamer head and the reamer shaft.

Example 13 can include, or can optionally be combined with the subject matter of Example 12 to optionally include a container coupled to the removal tool to catch a reamer head separated from the reamer shaft by the forked wedge.

Example 14 can include or use subject matter such as a method of determining an intramedullary nail length, the method comprising: attaching an initial reamer head to a reamer shaft having markers indicating a plurality of system lengths from a distal tip of the initial reamer head to the markers; reaming a canal into a bone using the initial reamer head; advancing the reamer shaft into the canal; reading a system length of the distal tip of the initial reamer head to an end of the canal from one of the markers; and selecting a size of an intramedullary device from the read system length.

Example 15 can include, or can optionally be combined with the subject matter of Example 14 to optionally include forming a pilot hole in the bone before reaming; inserting a guidewire into the pilot hole; and advancing the reamer shaft and the initial reamer head along the guidewire to form the canal.

Example 16 can include, or can optionally be combined with the subject matter of Examples 14 and 15 to optionally include removing the reamer shaft and the initial reamer head from the canal; removing the initial reamer head from the reamer shaft; attaching a different reamer head to the reamer shaft, the different reamer head having a head length equal to a head length of the initial reamer head such that the plurality of system lengths from a distal tip of the different reamer head to the markers is the same as the plurality of system lengths from a distal tip of the initial reamer head to the markers, wherein the initial reamer head and the different reamer head have different reaming diameters.

Example 17 can include, or can optionally be combined with the subject matter of Example 16 to optionally include using a wedge-shaped removal tool to remove the initial reamer head from the reamer shaft.

Example 18 can include, or can optionally be combined with the subject matter of Examples 14-17 markers comprising radiopaque markers.

Example 19 can include, or can optionally be combined with the subject matter of Example 18 to optionally include radiopaque markers comprising a plurality of circumferential rings extending from the reamer shaft.

Example 20 can include, or can optionally be combined with the subject matter of Example 18 to optionally include reading the system length of the distal tip of the initial reamer head to an end of the canal from one of the markers using a fluoroscopy system.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced.

These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A system for measuring an intramedullary length for an orthopedic implant, the system comprising:
a reamer shaft comprising:
a proximal end having a drive coupler;
a distal end having a reamer coupler; and
markers located along the reamer shaft proximate the proximal end; and
a first reamer head comprising:
a head coupler configured to connect with the reamer coupler; and
one or more cutting flutes located distal to the head coupler;
wherein the markers provide an indication of lengths of the system from a distal tip of the cutting flutes to each of the markers.

2. The system of claim 1, wherein the first reamer head comprises:
a first length corresponding to the length of the system minus a length of the reamer shaft; and
a first cutting diameter.

3. The system of claim 2, further comprising a second reamer head comprising:
a second length approximately equal to the first length; and
a second cutting diameter different from the first cutting diameter.

4. The system of claim 1, wherein:
the reamer coupler comprises a bulbous projection; and
the head coupler comprises a slot shaped to inversely match the bulbous projection.

5. The system of claim 4, wherein the bulbous projection includes a split.

6. The system of claim 1, wherein:
the reamer coupler comprises a socket having a ridge; and
the head coupler comprises a plurality of flexible fingers configured to axially engage the ridge of the socket.

7. The system of claim 6, wherein:
the reamer coupler further comprises a circumferentially lobed extension of the socket; and
the head coupler further comprises a key from which the plurality of flexible fingers extend, the key shaped to mate with the lobed extension and prevent relative rotation therebetween.

8. The system of claim 1, wherein the reamer shaft and the first reamer head include axially aligned lumens.

9. The system of claim 1, wherein the reamer coupler comprises a separate piece from the reamer shaft mechanically joined to the reamer shaft.

10. The system of claim 1, wherein the markers are radiopaque.

11. The system of claim 10, wherein the markers comprise a plurality of circumferential ribs extending from the reamer shaft.

12. The system of claim 1, further comprising a removal tool comprising:
a forked wedge that is shaped to be inserted into a gap between the reamer head and the reamer shaft.

13. The system of claim 12, further comprising a container coupled to the removal tool to catch a reamer head separated from the reamer shaft by the forked wedge.

14. A method of determining an intramedullary nail length, the method comprising:
attaching an initial reamer head to a reamer shaft having markers indicating a plurality of system lengths from a distal tip of the initial reamer head to the markers;
reaming a canal into a bone using the initial reamer head;
advancing the reamer shaft into the canal;
reading a system length of the distal tip of the initial reamer head to an end of the canal from one of the markers; and
selecting a size of an intramedullary device from the read system length.

15. The method of claim 14, further comprising:
forming a pilot hole in the bone before reaming;
inserting a guidewire into the pilot hole; and
advancing the reamer shaft and the initial reamer head along the guidewire to form the canal.

16. The method of claim 14, further comprising:
removing the reamer shaft and the initial reamer head from the canal;
removing the initial reamer head from the reamer shaft;
attaching a different reamer head to the reamer shaft, the different reamer head having a head length equal to a head length of the initial reamer head such that the plurality of system lengths from a distal tip of the different reamer head to the markers is the same as the plurality of system lengths from a distal tip of the initial reamer head to the markers, wherein the initial reamer head and the different reamer head have different reaming diameters.

17. The method of claim 16, further comprising using a wedge-shaped removal tool to remove the initial reamer head from the reamer shaft.

18. The method of claim 14, wherein the markers comprise radiopaque markers.

19. The method of claim 18, wherein the radiopaque markers comprise a plurality of circumferential rings extending from the reamer shaft.

20. The method of claim 18, further comprising reading the system length of the distal tip of the initial reamer head to an end of the canal from one of the markers using a fluoroscopy system.
